(19) United States
(12) Patent Application Publication
(10) Pub. No.: US 2010/0136531 A1
(43) Pub. Date: Jun. 3, 2010

(54) NUCLEIC ACID DETECTION USING LATERAL FLOW METHODS

(75) Inventors: Ian Garthwaite, New South Wales (AU); Philip A. Myers, New South Wales (AU); Christine M. Sadek, New South Wales (AU)

Correspondence Address: 3M INNOVATIVE PROPERTIES COMPANY, PO BOX 33427, ST. PAUL, MN, 55133-3427, US

(73) Assignee: TECRA INTERNATIONAL PTY LTD, Frenchs Forest, NSW (AU)

(21) Appl. No.: 12/296,536

(22) PCT Filed: Apr. 10, 2007
(86) PCT No.: PCT/IB2007/000923

§ 371 Date: Nov. 6, 2009

Related U.S. Application Data
(60) Provisional application No. 60/790,536, filed on Apr. 10, 2006.

(30) Foreign Application Priority Data
Apr. 10, 2006 (AU) 2006901847

(51) Int. Cl. C12Q 1/68

(52) U.S. Cl. 435/6

(57) ABSTRACT

Methods and kits for use in detecting a target nucleic acid in a sample are disclosed. In one particular application, the methods and kits allow for the detection of an undesirable micro-organism (e.g. Listeria, Salmonella or Enterobacteriaceae) in food or present on a food preparation surface.

### PRIORITY DOCUMENTS

The present application claims priority from:

Australian Provisional Patent Application No. 2006901847 entitled “Nucleic Acid Detection Method” and filed on 10 Apr. 2006; and U.S. Provisional Patent Application No. 60/790,536 entitled “Nucleic Acid Detection Method” and filed on 10 Apr. 2006. The entire content of both of these applications is hereby incorporated by reference.

### FIELD OF THE INVENTION

The present invention relates to methods and kits for use in detecting a target nucleic acid in a sample. In one particular application, the invention allows for the detection of an undesirable micro-organism (e.g. Listeria, Salmonella or Enterobacter) in food or present on a food preparation surface.

### BACKGROUND TO THE INVENTION

In recent years, the number of reported outbreaks of food poisoning caused by micro-organisms has increased worldwide. These food pathogens can be found as contaminants in a wide variety of foods including meat products (e.g. red meat, poultry and seafood), egg products, dairy products (e.g. cheese, milk and ice-cream), confectionery, and fruit and vegetables as well as in the food processing environment (e.g. a food preparation surface). Salmonella and Listeria, in particular, are recognised by the food safety regulators of most countries as the cause of significant contamination of food, and many of these food safety regulators require environmental and end-testing for these bacteria. Consequently, it is common practice to regularly check both food products and food processing environments for contamination by such micro-organisms. Similar testing is also conducted within other industries, such as the pharmaceutical and cosmetics manufacturing industries.

Testing for micro-organisms generally involves obtaining a sample such as a food sample, a swab from the area being tested, or samples taken from floor sweepings, waste and process water or filtered air, transferring the sample to a pre-enrichment or enrichment medium to enhance recovery and repair of damaged micro-organisms, subsequently conducting one or two additional selective enrichment steps to increase the numbers of the micro-organisms of interest, and thereafter testing for the presence of particular micro-organisms in the medium using traditional culturing methods or rapid methods such as immunoassays.

Rapid methods of testing for Listeria and Salmonella have been incorporated into systems supplied by the present applicant. In one example known as the UNIQUE™ system, described in Australian patent specification No 610925, the system involves firstly transferring a sample to a pre-enrichment medium for 16 hours, and then transferring a small aliquot of the pre-enrichment medium to a first tube into which a dipstick coated with antibodies against the micro-organism of interest (e.g. anti-Salmonella antibodies) is inserted for 20 minutes, during which time any micro-organisms present in the first tube are captured onto the dipstick surface. Thereafter, the system involves washing the dipstick in a second tube before transferring the dipstick to a third tube containing growth medium, and culturing any micro-organisms bound to the dipstick to multiply on the dipstick surface until present in a sufficient number to permit detection. For Salmonella, this culturing stage typically takes about 4 hours. After the culturing stage, the UNIQUE™ system then involves incubating the dipstick for 30 minutes in a fourth tube containing antibodies against the micro-organism of interest labelled with an enzyme (e.g. horseradish peroxidase or alkaline phosphatase) which bind to any micro-organisms present on the dipstick, then washing the dipstick in a fifth tube (i.e. to remove excess or unbound labelled antibodies) and, lastly, transferring the dipstick to a sixth tube containing a chromogen precursor for the enzyme label. If micro-organisms of interest are present, a chromogen (generally, purple in colour) is produced from the precursor and this appears as a coloured region on the dipstick.

This UNIQUE™ system has proven to be very reliable for a number of micro-organisms such as Listeria and Salmonella. However, that said, the present applicant recognised that improvements to achieve a system that was more convenient and involve less user time, would increase reliability by improving, for example, compliance with the optimal times and conditions (e.g. temperature) for the various incubation/culturing stages. To this end, the UNIQUE™ system has been automated, and the automated UNIQUE PLUS™ system is described in the applicant's co-pending Australian patent application No 2002333050.

Due to the often serious consequences or repercussions of a “positive” test result for micro-organisms in a sample, it is often desirable to conduct confirmatory tests on the same or similar sample. Presently, for the UNIQUE™ and UNIQUE PLUS™ systems, such confirmatory tests are performed by simply plating out onto agar a sample aliquot from the first or third tubes mentioned above, and testing any growing micro-organisms for biochemical and morphological characteristics. This process may be prone to error and can also be laborious and cause significant time delays (e.g. confirmatory results may take up to 48 to 72 hours using this process). Moreover, for some micro-organism detection systems, a positive test result may only be indicative of the presence of a micro-organism from a particular genus, whereas it may be preferable or desirable to identify a particular species (e.g. for foods contaminated with Listeria, product recall may only be mandated where the contamination is by the human pathogen, Listeria monocytogenes). The present applicant describes hereinafter, simple, quick (e.g. about 1 to 4 hours) and reliable methods for detecting a micro-organism such as a food pathogen, that can be readily used with samples obtained from a UNIQUE™ or UNIQUE PLUS™ system test (e.g. a sample aliquot from the first or third tubes mentioned above) or other suitable sample, so as to provide a confirmatory result, and which may also be performed in a manner whereby the identity of a particular micro-organism species can be revealed. The methods described are also suitable for use in screening assays.

### SUMMARY OF THE INVENTION

Thus, in a first aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to said second label and said control region being provided with a control agent; and
(v) detecting any binding of constituents of the buffered product at said test region and at said control region.

The detection of binding at the test region provides a result showing the presence in the sample of the micro-organism intended to be detected.

The control agent may specifically bind to the first agent, in which case the detection of binding at the control region provides a result showing that the microparticles have successfully flowed across the substrate and that the microparticle-bound first agent is able to be bound by the control agent.

Binding at the test region and control region is conveniently detected by viewing the appearance of colour, as can be provided by the microparticles.

Preferably, the control region is located between the test region and the distal end of the lateral flow device.

The method can be readily varied such that the first label of the first primer sequence is omitted and replaced with labelled deoxyribonucleotide triphosphates (dNTPs).

Thus, in a second aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels;
(iii) diluting an amount of the amplification product of step (ii) in a suitable buffer solution comprising microparticles labelled with a first agent which specifically binds to one of said first label and allowing said first agent to bind to said first label present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to the second label and said control region being provided with a control agent; and
(v) detecting any binding of constituents of the buffered product at said test region and at said control region.

### BRIEF DESCRIPTION OF THE FIGURES

FIG. 1 provides a diagrammatic representation of a lateral flow device suitable for use in the method of the invention. The representation shows that the device (1) comprises a sample pad (2), a membrane (3), an absorption pad (4), and backing card (5). A test region (6) and control region (7) are shown adjacent to the absorption pad (4). The test region (6) provides a test result and the control region (7) provides a positive control result.

### DETAILED DESCRIPTION OF THE INVENTION

The present invention provides methods for the detection of a micro-organism present in a sample. In particular, said methods are intended for the detection of micro-organisms found in food such as bacteria (e.g. Listeria, Salmonella, Enterobacter, Escherichia, Legionella, Bacillus, Pseudomonas, Staphylococcus, Campylobacter and Helicobacter), however, the method is also suitable for detecting other types of micro-organisms which may be found in a food, water or other environmental sample such as viruses, yeasts, moulds and protozoa (e.g. Cryptosporidium).

The method of the invention, in a first aspect, comprises the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region provided with a second agent which specifically binds to said second label and said control region being provided with a control agent; and
(v) detecting any binding of constituents of the buffered product at said test region and at said control region.

The sample may be any suitable sample including, for example, a food sample, a sample prepared from a swab of a food preparation surface, a waste or process water sample, and a micro-organism culture or enrichment sample (e.g. a sample aliquot from the first or third tubes of a UNIQUE™ system test).

It has been found that samples containing a micro-organism intended to be detected do not always require any step of isolating nucleic acid from the micro-organism prior to the amplification of step (ii). Instead, the sample may only need to be treated, preferably by heating (e.g. at a temperature in the range of 85 to 100° C.), so as to cause release of micro-organism nucleic acid (e.g. by lysis) and, preferably, denaturation (i.e. “strand melting”) of any double stranded nucleic acid (e.g. dsDNA) into single stranded nucleic acid (e.g. ssDNA). Step (i) may not necessarily be conducted by the same party who conducts the remainder of the method steps (e.g. a sample collector may heat the sample to cause release of nucleic acid from any micro-organism present, before delivering the sample to a laboratory).

The amplification step (ii) may be performed using any of the methods well known to persons skilled in the art. Preferably, the amplification is performed using a standard polymerase chain reaction (PCR) amplification method using a pair of primer sequences (i.e. first and second primer sequences) defining the 5′ and 3′ ends of a target nucleotide sequence. However, in some circumstances, it may be preferred to perform the amplification step using a “nested” PCR amplification method using a further, “outside”, pair of primer sequences (i.e. first and second outside primer sequences).

By selecting primer sequences that are species specific, the method can be performed in a manner whereby the identity of a particular micro-organism species present in the sample can be revealed.

The first and second primer sequences are preferably selected such that amplicons generated during the amplification step (ii) are in the range of 40 to 3000 nucleotides in length, more preferably 50 to 1500 nucleotides in length. Generally, the shorter the amplicon, the more rapidly the amplification step (ii) can be completed. It will be well understood by persons skilled in the art that, where appropriate, the first and second primers may be degenerate primers or, otherwise, may include multiple primers as required, to ensure detection of the target nucleotide sequence.

In the method of the first aspect, the first and second primer sequences are labelled with first and second labels, respectively. Preferably, the first and second labels are selected from haptens such as, for example, biotin, fluorescein derivatives (e.g. FITC), rhodamine derivatives (e.g. TAMRA), Cascade Blue, Lucifer yellow, 5-bromo-2-deoxyuridine (BrdU), dinitrophenol (DNP), digoxygenin (DIG), and short peptide label sequences (i.e. short peptides against which specific antibodies can be raised). More preferably, the first label is biotin and the second label is DNP, in which case, amplicons generated during the amplification step (ii) are labelled with both biotin and DNP.

Following the amplification step (ii), an amount of the amplification product is diluted in a suitable buffer solution. This buffer comprises microparticles labelled with a first agent which specifically binds to the first label. This step (iii) can simply involve the direct dilution of the amplification product into a prepared buffer solution comprising the said microparticles, or it can otherwise involve a step-wise dilution process wherein the amplification product is finally diluted in the said suitable buffer solution comprising the microparticles. A particular embodiment of such a step-wise dilution process involves, firstly, adding an amount of the amplification product to a suitable buffer solution that lacks said microparticles, and thereafter adding said microparticles to the amplification product-buffer solution composition. Conveniently, the addition of the microparticles to the amplification product-buffer solution composition can be achieved by placing the amplification product-buffer solution composition into contact with a receptacle or surface onto which said microparticles have been dried, such that the microparticles become suspended in the amplification product-buffer solution composition thereby forming the required suitable buffer solution comprising said microparticles.

Step (iii) is conducted for a sufficient period of time to allow the first agent to bind to said first label present in the amplification product. Preferably, step (iii) is conducted for a duration in the range of 0.1 to 5 minutes, more preferably for 0.2 to 1 minutes.

The microparticles may be composed of a wide variety of substances, but are preferably composed of one or more substantially inert substances such as gold, silica, selenium, polystyrene, melamine resin, polymethacrylate, styrene/divinylbenzene copolymer, and polyvinyltoluene. The microparticles are preferably non-porous. The microparticles may comprise a substance to allow for visualisation of results at the test region and control region on the lateral flow device. Conveniently, such a substance will be a dye or other coloured substance to allow for visualisation with the unaided eye, however alternatively, the substance may be, for example, a label substance allowing visualisation through the generation of a coloured substance (e.g. an enzyme or other catalytic-label) or by fluorescence, luminescence or magnetic interactions (e.g. using a fluorimeter, luminometer or magnetic induction). The microparticles may be of a diameter size in the range of 0.002 to 5 μm. Preferably, the microparticles are gold microparticles having a diameter size in the range of 0.002 to 0.25 μm (i.e. 2 to 250 nm), more preferably 0.01 to 0.06 μm (i.e. 10 to 60 nm), and most preferably having an average diameter size of 0.04 μm (i.e. 40 nm). Suitable polystyrene microparticles include those having a diameter size in the range of 0.1 to 5 μm.

The first agent is selected from agents capable of specifically binding or reacting with the first label. As such, the first agent may be an antibody, antibody fragment (e.g. Fab, F(ab′)₂ and scfv fragments), receptor or other binding partner. The first agent itself may be conjugated to an enzyme or catalytic substance allowing visualisation through the generation of a detectable product following addition of a suitable substrate. Where the first label is biotin, the first agent may be streptavidin or avidin, but more preferably, is an anti-biotin antibody.

The buffered product of step (iii) is applied at or adjacent to the proximal end of a lateral flow device. The lateral flow device comprises a substrate which allows constituents of the buffered product to wick or flow laterally towards a distal end of the lateral flow device. Generally, the lateral flow device is in the form of a strip of the substrate (e.g. of 4-8 mm×40-80 mm in dimensions). Preferably, the substrate is composed of nitrocellulose membrane, polyvinylidene fluoride (PVDF), nylon or a single porous material matrix (e.g. Fusion 5 (Whatman, Middlesex, UK), and as described in US patent specification No 2006/0040408).

The lateral flow device is provided, at or adjacent to the distal end, with a test region and a control region. The test region is provided with a second agent which specifically binds to said second label and said control region is provided with a control agent. Agents may be conjugated or adsorbed to proteins, microparticles or other substances to allow for immobilisation of agents to the test substrate.

The test region provides a test result. That is, the test region binds and immobilises amplicons (wherein each amplicon should be bound to a microparticle) and thereby provides a result showing the presence or absence in the sample of the micro-organism intended to be detected. A “positive” test result (i.e. a test result indicating the presence of the micro-organism in the sample) is preferably indicated by the appearance of a visible colour signal, as provided by the microparticles, at the test region. Where the microparticles are gold microparticles, the visible colour signal will be a pinkish-red colour.

The control region is a region on the lateral flow device separate from the test region. Preferably, the control region is located between the test region and the distal end of the lateral flow device. The control region provides a positive control result. As described below, this positive control result can show that the microparticles have successfully flowed across the substrate or, otherwise, indicate that the amplification of step (ii) was successful. Optionally, the control region can comprise first and second sub-regions, the first sub-region able to provide a positive control result showing that the microparticles have successfully flowed across the substrate, and the second sub-region able to indicate that the amplification of step (ii) was successful.

Thus, in the simplest case, the control region is provided with a control agent which specifically binds to the first agent, in which case the control region binds and immobilises microparticles and thereby provides a result to indicate whether the microparticles have successfully flowed across the substrate. The amount of microparticles present in the buffer solution of step (ii) may therefore be sufficient to provide an amount of unbound microparticles (and/or microparticles bound to unincorporated first primers) for binding to such a control region. However, the amount of microparticles present in the buffer solution may otherwise be sufficient if it provides, in addition to or as an alternative to an amount of unbound microparticles, an amount of bound microparticles (i.e. bound to doubly labelled amplicons) which provides an excess to that amount which may be bound by the test region (i.e. thereby providing an amount of bound microparticles which may flow past the test region to the control region). Where the control region comprises a control agent that is the same or functionally equivalent to the first label, a positive control result indicates that binding between the first agent and first label ought to have been successful.

A positive control for the amplification of step (ii) can be particularly valuable where the amplification might be performed with the presence of potentially inhibitory molecules (e.g. as might be found in the sample). The positive control for the amplification of step (ii) can be conducted in a separate amplification vessel by performing an amplification of a control nucleotide sequence (e.g. a sequence of approximately equal length to the target nucleotide sequence) provided on a control nucleic acid in parallel with the amplification of step (ii), and thereafter combining the product of the separate amplifications prior to step (iii). However, preferably, the positive control for the amplification involves the inclusion, in step (ii) of a control nucleic acid providing a control nucleotide sequence, and a pair of third and fourth primer sequences defining the 5′ and 3′ ends of the control nucleotide sequence. The control nucleic acid can, therefore, be added into the amplification mixture of step (ii) or, otherwise, introduced into the said sample before or after the treatment of step (i). Preferably, the third and fourth primers will preferably have similar melting temperature (Tm) and priming characteristics as the first and second primers so as to allow for the same annealing temperature and amplification time to be used. The third and fourth primer sequences are labelled with third and fourth labels, respectively, which preferably both differ from the first and second labels (e.g. all of biotin, DNP, FITC and DIG might be used), although it may be possible for the first and third labels to be the same (or otherwise functionally equivalent), in which case the microparticles labelled with a first agent in step (iii) ought to bind to amplicons generated from the first and second primer sequences as well as amplicons generated from the third and fourth primer sequences. However, where the first and third labels are different, the inclusion of a positive control for amplification necessitates adding to the buffer solution used in step (iii) microparticles labelled with a third agent which specifically binds to the third label. The control region on the lateral flow device, in this case, will comprise a control agent which specifically binds to the fourth label.

Accordingly, step (ii) of the method of the first aspect, may comprise:
providing a control nucleic acid, and co-amplifying
a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels, and
a control nucleotide sequence present on said control nucleic acid, wherein said amplification of the control sequence comprises the use of a pair of third and fourth primer sequences defining the 5′ and 3′ ends of said control sequence, said third primer sequence being labelled with a third label (which may be the same as, or functionally equivalent to, the first label) and said fourth primer sequence being labelled with a fourth label such that any amplification of the control sequence generates an amplicon labelled with both third and fourth labels.

A positive control result is preferably indicated by the appearance of a visible colour signal, as provided by the microparticles, at the control region. Where the microparticles are gold microparticles, the visible colour signal will be a pinkish-red colour.

The lateral flow device may further comprise a sample pad at the proximal end and/or an absorption pad located at the distal end, to assist in the flow of the constituents of the buffer product of step (iii) across the substrate. Moreover, the lateral flow device may be provided with a supporting means for the substrate such as, for example, a sheet of cardboard or rigid polymer, to which the substrate may be affixed. Additionally, the lateral flow device may be provided with two or more identical or similar devices in any suitable container (e.g. a tray) for holding the devices in a multi- or, even, macro-array for use.

The step of detecting any binding of constituents of the buffered product of step (iii) at the test region and the control region is most preferably conducted by simply viewing the appearance of a visible colour signal, as provided by the microparticles, with the unaided eye. However, the appearance of colour or the intensity of the colour may be measured using a light or reflectance detector (e.g. a charge coupled device (CCD) or photopic sensor). The intensity of the colour at the test region may be used as a semi-quantitative measure of the amount of the micro-organism present in the sample. Certain microparticles may also be used as labels that absorb or emit detectable radiation (e.g. light of specific wavelengths).

As mentioned above, the amplification step (ii) may be performed using a nested PCR amplification method. In such an embodiment, the nested PCR amplification is preferably conducted in a single amplification vessel (e.g. tube) containing both “inside” and “outside” pairs of primer sequences. The inside pair of primer sequences correspond to the first and second primer sequences, whereas the outside pair of primer sequences are provided by first and second outside primer sequences. The first and second outside primer sequences are selected to enable amplification of sequences flanking the target nucleotide sequence; they will generally be unlabelled. Nested PCR amplification offers the possibility of increased specificity and sensitivity since the detection of amplicons caused by mispriming (i.e. amplicons generated from nucleic acid other than that of the micro-organism intended to be detected) is more likely to be avoided. In addition, the amplification step (ii) may employ multiplex PCR or, otherwise, use degenerate primers to ensure specific detection of the target micro-organism(s).

The method of the first aspect can be readily varied to enable simultaneous detection of a micro-organism(s) belonging to a particular family (e.g. Listeriaceae, Enterobacteriaceae, Staphylococcaceae, Bacillaceae, Legionellaceae, Pseudomonadaceae, Campylobacteraceae and Helicobacteraceae) as well as, more specifically, a micro-organism of a particular genus (e.g. Listeria, Salmonella, Enterobacter, Escherichia, Legionella, Bacillus, Pseudomonas, Staphylococcus, Campylobacter and Helicobacter) within the family; or similarly, simultaneous detection of a micro-organism(s) belonging to a particular genus as well as, more specifically, a micro-organism of a particular species of that genus. For example, using a genus-specific primer pair (e.g. a Listeria spp.-specific primer pair) and a species-specific primer pair (e.g. a L. monocytogenes-specific primer pair). In this embodiment, the first and second primer sequences (comprising the species-specific primer) are labelled with, respectively, first and second labels, and third and fourth primer sequences (comprising the genus-specific primers) are labelled with, respectively, third and fourth labels which preferably both differ from the first and second labels (e.g. all of biotin, DNP, FITC and DIG might be used), although it may be possible for the first and third labels to be the same or functionally equivalent (i.e. such that the first and third labels are both able to be bound by the first agent). The amplification would preferably be conducted in a single amplification vessel (i.e. a multiplex reaction) and, as such, the third and fourth primer sequences will preferably have similar melting temperature (Tm) and priming characteristics so as to allow for the same annealing temperature and amplification time to be used. However, the amplification may otherwise be conducted in separate amplification vessels with the product of the separate amplifications being combined prior to step (iii). The first and third labels may be the same (or otherwise functionally equivalent), in which case the microparticles labelled with a first agent in step (iii) ought to bind to amplicons generated from the first and second primer sequences as well as amplicons generated from the third and fourth primer sequences. However, where the first and third labels are different, the method necessitates adding to the buffer solution used in step (iii), microparticles labelled with a third agent which specifically binds to the third label. The method also necessitates providing on the lateral flow device, an additional test region provided with a fourth agent which specifically binds to the fourth label. The additional test region thereby binds and immobilises amplicons generated from the third and fourth primer sequences.

The method of the first aspect can also be readily varied to enable simultaneous detection of more than one type of micro-organism (e.g. Listeria and Salmonella). In this embodiment, the amplification would preferably be conducted in a single amplification vessel containing the pair of first and second primer sequences and a further pair of third and fourth primer sequences. The first and second primer sequences are selected to amplify a target nucleotide sequence of a first micro-organism (e.g. Listeria), whereas the third and fourth primer sequences are selected to amplify a target nucleotide sequence of a second micro-organism (e.g. Salmonella). The third and fourth primer sequences are labelled with third and fourth labels, respectively, which preferably both differ from the first and second labels (e.g. all of biotin, DNP, FITC and DIG might be used), although it may be possible for the first and third labels to be the same (or otherwise functionally equivalent), in which case the microparticles labelled with a first agent in step (iii) ought to bind to amplicons generated from the first and second primer sequences as well as amplicons generated from the third and fourth primer sequences. However, where the first and third labels are different, the method necessitates adding to the buffer solution used in step (iii), microparticles labelled with a third agent which specifically binds to the third label. The amplification would preferably be conducted in a single amplification vessel (i.e. a multiplex reaction) and, as such, the third and fourth primer sequences will preferably have similar melting temperature (Tm) and priming characteristics so as to allow for the same annealing temperature and amplification time to be used. However, the amplification may otherwise be conducted in separate amplification vessels with the product of the separate amplifications being combined prior to step) (iii). The method also necessitates providing on the lateral flow device, an additional test region provided with a fourth agent which specifically binds to the fourth label. The test region binds and immobilises amplicons generated from the first and second primer sequences (thereby indicating the presence of the first micro-organism), whereas the additional test region binds and immobilises amplicons generated from the third and fourth primer sequences (thereby indicating the presence of the second micro-organism).

In an alternative to the method of the first aspect, the invention can be readily varied such that the first label of the first primer sequence is omitted and replaced with labelled deoxyribonucleotide triphosphates (dNTPs) such as, for example, labelled 2′-deoxyadenosine 5′-triphosphate (dATPs) and/or labelled 2′-deoxythymidine triphosphate (dTTPs).

Thus, in a second aspect, the present invention provides a method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to the other of said first and second labels which is not bound by said first agent and said control region being provided with a control agent; and
(v) detecting any binding of constituents of the buffered product at said test region and at said control region.

For the purpose of removing any doubt, it is to be understood that throughout the specification, reference to said first primer sequence may be a reference to the “forward” or “reverse” primer sequences. Similarly, reference to said second primer sequence may be a reference to the “forward” or “reverse” primer sequences. However, necessarily, the said first and second primer sequences, together, define the 5′ and 3′ ends of the target nucleotide sequence of the micro-organism.

The control region of the method of the second aspect, preferably provides a positive control for the amplification of step (ii). As such, the third primer sequence is labelled with a third label that is the same as the first label (or otherwise functionally equivalent) and the fourth primer is labelled with a fourth label which differs from the first (and third) and second labels, in which case the microparticles labelled with a first agent in step (iii) ought to bind to amplicons generated from the first and second primer sequences as well as amplicons generated from the third and fourth primer sequences. The control region on the lateral flow device, in this case, will comprise a control agent which specifically binds to the fourth label. The control region binds and immobilises amplicons generated from the third and fourth primer sequences, thereby indicating that the amplification of step (ii) was successful. The control region can also indicate that microparticles have successfully flowed across the substrate and that microparticle-bound third agent has been able to bind to the third label. If the first and third labels are the same (or functionally equivalent), the control region may also indicate whether the microparticle-bound first agent has been able to bind to the first label.

Accordingly, step (ii) of the method of the second aspect may comprise:
providing a control nucleic acid, and co-amplifying
a target nucleotide sequence present on said micro-organism nucleic acid, said target sequence being unique or otherwise characteristic of said micro-organism, and wherein said amplification of the target sequence comprises the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label such that any amplification of the target sequence generates an amplicon labelled with both first and second labels, and
a control nucleotide sequence present on said control nucleic acid, wherein said amplification of the control sequence comprises the use of a pair of third and fourth primer sequences defining the 5′ and 3′ ends of said control sequence, wherein the amplification of the control sequence utilises dNTPs labelled with the first label and said fourth primer sequence is labelled with a fourth label such that any amplification of the control sequence generates an amplicon labelled with both third and fourth labels.

In a third aspect, the present invention provides a kit for the detection of a micro-organism present in a sample, said kit comprising:
a pair of first and second primer sequences defining 5′ and 3′ ends of a target nucleotide sequence that is unique or otherwise characteristic of said micro-organism, said first primer sequence being labelled with a first label and said second primer sequence being labelled with a second label;
a buffer solution comprising microparticles labelled with a first agent which specifically binds to said first label; and
a lateral flow device comprising a substrate with a test region and a control region, said test region being provided with a second agent which specifically binds to said second label and said control region being provided with a control agent which specifically binds to the first agent.

In a fourth aspect, the present invention provides a kit for the detection of a micro-organism present in a sample, said kit comprising:
deoxyribonucleotide triphosphates (dNTPs) labelled with a first label (e.g. a mix of dNTPs including labelled dATPs);
a pair of first and second primer sequences defining 5′ and 3′ ends of a target nucleotide sequence that is unique or otherwise characteristic of said micro-organism, said second primer sequence being labelled with a second label;

a buffer solution comprising microparticles labelled with a first agent which specifically binds to said first label; and
a lateral flow device comprising a substrate with a test region and a control region, said test region being provided with a second agent which specifically binds to said second label and said control region being provided with a control agent which specifically binds to the first agent.

The kit of the third or fourth aspects may further comprise other components such as wash solutions and blocking reagents, a control nucleic acid (e.g. an oligonucleotide) and a pair of primer sequences defining the 5′ and 3′ ends of a control nucleotide sequence, and a suitable polymerase enzyme (e.g. Taq polymerase).

The method of the present invention can be readily adapted to detect nucleic acids from non-micro-organism sources that may be suspected of being present in a particular sample, for example, human nucleic acids in blood samples (e.g. to enable, for example, genotyping of an individual) and nucleic acids from plants and other animals (e.g. for the detection of food allergens such as peanut, egg and shellfish allergens).

Thus, in a fifth aspect, the present invention provides a method for the detection of a nucleic acid in a sample, said method comprising the steps of:
(i) treating said sample so as to cause release of nucleic acid from any cell (e.g. a mammalian, insect or plant cell) or other nucleic acid-containing structure (e.g. a viral capsid) present in the sample;
(ii) amplifying a target nucleotide sequence present on said nucleic acid, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to said second label and said control region being provided with a control agent; and
(v) detecting any binding of constituents of the buffered product at said test region and at said control region.

And, in a sixth aspect, the present invention provides a method for the detection of a nucleic acid in a sample, said method comprising the steps of:
(i) treating said sample so as to cause release of nucleic acid from any cell or other nucleic acid-containing structure present in the sample;
(ii) amplifying a target nucleotide sequence present on said nucleic acid, comprising the use of a pair of first and second primer sequences defining the 5′ and 3′ ends of said target sequence, wherein the amplification of the target sequence utilises deoxyribonucleotide triphosphates (dNTPs) labelled with a first label and said second primer is labelled with a second label, such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to the other of said first and second labels which is not bound by said first agent and said control region being provided with a control agent; and
(v) detecting any binding of constituents of the buffered product at said test region and at said control region.

As with the methods of the first and second aspects, the methods of the fifth and sixth aspects utilise a control region. The control region is a region on the lateral flow device separate from the test region. Preferably, the control region is located between the test region and the distal end of the lateral flow device. The control region provides a positive control result. In a manner equivalent to that described in relation to the methods of the first and second aspects above, this positive control result can show that the microparticles have successfully flowed across the substrate or, otherwise, indicate that the amplification of step (ii) was successful. Optionally, the control region can comprise first and second sub-regions, the first sub-region able to provide a positive control result showing that the microparticles have successfully flowed across the substrate, and the second sub-region able to indicate that the amplification of step (ii) was successful.

In the methods of the second and sixth aspects, the amplification of step (ii) may utilise 2′-deoxyuridine triphosphate (dUTP), which is preferably unlabelled, since the incorporation of dUTP into the amplicon provides a mechanism for degrading the generated amplicons by the use of a specific uracil degrading enzyme such as uracil-N-glycosylase (UNG). Examples of this enzyme that are well known to persons skilled in the art can be irreversibly heat-inactivated (e.g. HK™-UNG available from Epicentre Biotechnologies, Madison, Wis., United States of America). Therefore, where there may be concern that the amplification mix of step (ii) could be contaminated with extraneous nucleic acids (i.e. non-sample nucleic acids) which might include the target nucleotide sequence and thereby lead to a “false positive” result (e.g. contaminating amplicons from earlier amplifications that might be remaining on laboratory equipment or surfaces), then addition of an enzyme such as HK™-UNG to the sample prior to the amplification step (ii) should lead to the selective degradation of any extraneous nucleic acids comprising dUTPs. After a sufficient incubation time (e.g. 370-50° C. for about 15 minutes) to allow for any such extraneous nucleic acids to be degraded, the sample may be heated to irreversibly inactivate the HK™-UNG (e.g. by heating the sample to about 95° C.

In order that the nature of the present invention may be more clearly understood, preferred forms thereof will now be described with reference to the following non-limiting examples.

### EXAMPLES

### Example 1

### Detection of Listeria Using Labelled Primer Sequences

### Materials and Methods

### Sample

A sample was obtained from the third tube of a TECRA® UNIQUE PLUS™ Listeria test module (i.e. the third tube in the automated system described in Australian patent application No 2002333050 operated for Listeria detection).

### PCR Primers

Polymerase chain reaction (PCR) primers were selected to enable amplification of a nucleic acid sequence present in Listeria from a region of the 16s rRNA gene. The nucleotide sequences of the primers are:

(SEQ ID NO: 1)

| | |
|---|---|
| Forward primer: | 5′-GCGTGCCTAATACATGCAAG-3′ |

(SEQ ID NO: 2)

| | |
|---|---|
| Reverse primer: | 5′-ACCTCGCGGCTTCGCGAC-3′ |

The labelled and desalted primers were obtained from Sigma Proligo (Boulder, Colo., USA). The forward primer was labelled at the 5′ end with fluorescein while the reverse primer was labelled at the 5′ end with biotin. PCR amplification with these primers produced an amplicon of approximately 1234 nucleotides (e.g. L. monocytogenes 4b) in length.

### Lateral Flow Device

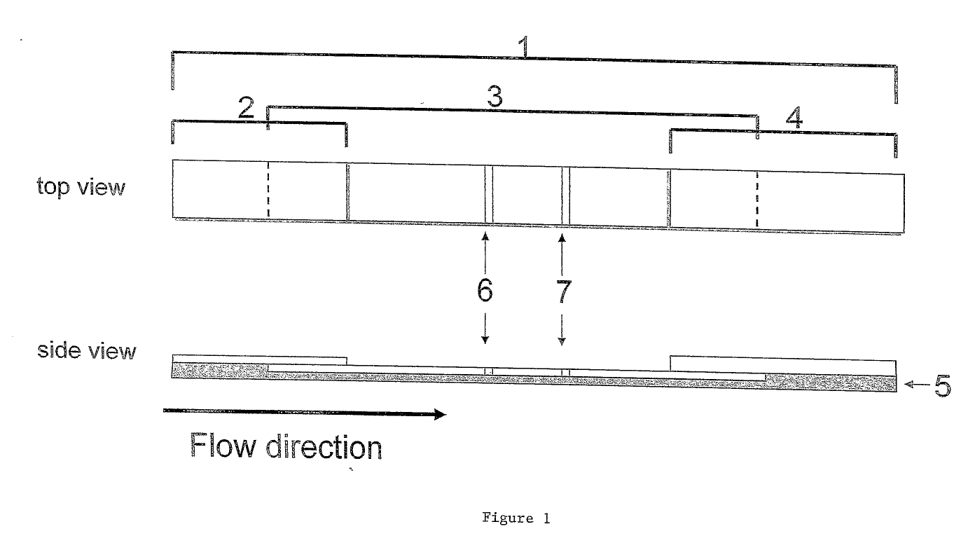

A lateral flow device as shown in FIG. 1 was prepared using a strip of nitrocellulose membrane (Immunopore FP, Whatman) of 5 mm×60 mm in dimensions. A sample pad (Arista Biologicals, Allentown, Pa., USA) was applied to the strip to allow loading of the buffered assay sample. At the distal end of the device, an absorbent pad comprising cotton fibre (Arista Biochemicals, Allentown, Pa., USA) was adhered to draw the flow of the buffered assay sample across the membrane. A test line and a positive control line were made on the membrane. The test line was prepared by adsorbing 2.3 anti-FITC monoclonal antibodies (Sigma-Aldrich, St. Louis, Mo., USA) to the membrane in a thin line across the width of the membrane. The positive control line was placed between the test line and the distal end of the device, and was prepared by adsorbing bovine serum albumin conjugated to biotin (Sigma-Aldrich) in a thin line across the width of the membrane. The entire lateral flow device was constructed by applying the membrane and sample and absorbent pads onto an adhesive backing card (Millenia Diagnostics, San Diego, Calif., USA).

### Amplification

PCR amplification was conducted in accordance with methods well known to persons skilled in the art. With the primers described above (i.e. SEC, ID NO: 1 and 2), the PCR amplification was conducted as follows:
(i) 1 μl of sample was inoculated using a sterile inoculation loop into 50 μl of PCR mix (comprising a final concentration of 0.5 μM of each primer, 20 units/ml Taq DNA polymerase, 200 μM dATP, 200 μM dCTP, 200 μM dGTP, and 200 μM dTTP, in a buffer of 1.5 mM MgCl₂, 10 mM Tris-HCl, 50 mM KCl, pH 8.3 at 25° C.);
(ii) Using a standard thermal cycler PCR machine, the inoculated PCR mix was subjected to an initial heating step of 94° C. for 5 minutes; and
(iii) 35 cycles of:
   a. melting step, 94° C. for 15 seconds,
   b. annealing step, approximately 58° C. for 20 seconds, and
   c. elongation step, 72° C. for 2 minutes.

### Preparation of PCR Product

Just prior to application to the lateral flow device, a 5 μl aliquot of the PCR product was mixed with 100 μl running buffer (phosphate buffered saline, pH 7.5 and 0.05% Tween-20) and 20 μl of gold microparticles (OD530=10.2) pre-adsorbed to goat anti-biotin (Alchemy Laboratories, Dundee, UK) that specifically bind to biotin.

### Assaying on the Lateral Flow Device

The buffered assay sample, comprising the entire 132 μl aliquot of the buffered PCR product/gold microparticle mixture, was loaded onto the sample pad of the lateral flow device as described above. The constituents of the mixture were allowed to flow across the membrane for 5 minutes. The test line comprising anti-FITC antibodies “trapped” doubly labelled amplicons present in the mixture that were labelled with FITC. Doubly labelled amplicons were bound to gold microparticles, and thus, when trapped at the test line by anti-FITC antibodies, generated a pinkish-red line. On the other hand, the positive control line trapped gold microparticles that either were not bound to amplicons or were not captured by the anti-FITC antibody of the test line. The positive control line confirmed that the gold microparticles flowed across the membrane and that the anti-biotin gold microparticles were capable of capturing biotin. Trapping of gold microparticles at the positive control line also generated a pinkish-red line.

### Results and Discussion

The sample was subjected to amplification as described above, and the amplification product diluted in a running buffer for assaying on the lateral flow device. As the origin of the sample (i.e. a third tube from an automated UNIQUE PLUS™ detection system being operated for Listeria detection) generated a positive result indicating the presence of Listeria, it was expected that the assay of this example would provide a confirmatory positive result. Following loading of the lateral flow device, two pinkish-red lines appeared on the membrane after about two minutes, thereby indicating the presence of Listeria in the sample. From the initial heating of the sample to the appearance of the two pinkish-red lines on the lateral flow device, took about 220 minutes (i.e. less than 4 hours). Comparative samples from pure cultures containing Salmonella only, assayed in parallel, produced no colour at the test line indicating a negative result for Listeria.

### Example 2

### Detection of Listeria Using Labelled dNTPs

### Materials and Methods

### Sample

A sample was obtained from the third tube of a TECRA® UNIQUE PLUS™ Listeria test module (i.e. the third tube in the automated system described in Australian patent application No 2002333050 operated for Listeria detection).

### PCR Primers

Polymerase chain reaction (PCR) primers used were the same as that used in Example 1, however, in this case, the forward primer was labelled at the 5′ end with fluorescein while the reverse primer was unlabelled. Again, the primers were obtained from Sigma Proligo.

### Lateral Flow Device

A lateral flow device as described in Example 1 was prepared. The test line was prepared by adsorbing anti-FITC monoclonal antibodies (Sigma-Aldrich) to the membrane in a thin line. The positive control line was prepared by adsorbing BSA conjugated to biotin (Sigma-Aldrich) to the membrane in a thin line.

### Amplification

PCR amplification was conducted in accordance with standard methods using a dNTP mix comprising biotin-labelled dATP (NEBiolabs, Ipswich, Mass., USA). The PCR amplification was conducted as follows:
(i) 2 μl of sample was inoculated into 50 μl of PCR mix (comprising a final concentration of 0.5 μM of each primer, 20 units/ml Taq DNA polymerase, 32 μM biotinylated dATP, 168 μM dATP, 200 μM dCTP, 200 μM dGTP, and 200 μM dTTP, in a buffer of 1.5 mM MgCl₂, 10 mM Tris-HCl, 50 mM KCl, pH 8.3 at 25° C.);
(ii) Using a standard thermal cycler PCR machine, the inoculated PCR mix was subjected to an initial heating step of 94° C. for 5 minutes; and
(iii) 35 cycles of:
   a. melting step, 94° C. for 15 seconds,
   b. annealing step, approximately 58° C. for 20 seconds, and
   c. elongation step, 72° C. for 2 minutes.

### Preparation of PCR Product

The PCR product was prepared for loading onto a lateral flow device in the same manner as that described in Example 1.

### Assaying on the Lateral Flow Device

The entire 132 μl aliquot of the buffered PCR product/gold microparticle mixture was loaded onto a lateral flow device as described in Example 1. The constituents of the mixture were allowed to flow across the membrane for 5 minutes.

### Results and Discussion

In samples that contained Listeria, doubly labelled amplicons were detected at the test line. The control line indicated complete flow of the gold microparticles to the distal end of the membrane and the ability of the anti-biotin gold microparticles to be captured by biotin. Comparative samples not containing Listeria were negative.

The use of biotinylated dNTPs with one labelled primer and one unlabelled primer for the amplification of a specific nucleic acid sequence produced a doubly labelled amplicon that was readily detected. This method may serve to increase the sensitivity of the assay as there are more biotin molecules available to be bound by the anti-biotin antibodies of the gold microparticles. However, the possibility of obtaining a false positive result from the assay could increase if mispriming occurs with the inclusion of the labelled primer into the amplicon.

### Example 3

### Detection of Listeria Using Two Labelled Primers and a Single Layer Porous Matrix Material

### Materials and Methods

### Sample

A PCR product sample containing doubly labelled amplicons of the Listeria 16s rRNA gene was obtained as described in Example 1.

### Lateral Flow Device

Fusion 5 membrane (Whatman) was adhered in a strip of dimensions 5 mm×60 mm to an equally sized adhesive backing card (Millenia Diagnostics). A test line was made by applying a stripe of 2.3 μm polystyrene beads (Bangs Laboratories, Fishers, Ind., USA) onto which anti-FITC monoclonal antibodies (Sigma-Aldrich) had been adsorbed according to a protocol provided by Bangs Laboratories. Briefly, 1 mg of polystyrene beads was added to 100 μl adsorption buffer (phosphate buffered saline, pH 7.4). In a separate tube, 23 μg anti-FITC antibody was diluted in 100 μl adsorption buffer. The polystyrene beads solution was transferred to the tube containing the antibody and the combined solutions were mixed for 2 hours at room temperature followed by overnight mixing at 4° C. to allow the antibody to adsorb to the polystyrene beads. Following adsorption, the beads were centrifuged gently (2000×g) and the supernatant containing un-adsorbed antibody was removed. The beads were then stored in 100 μl storage buffer (phosphate buffered saline, pH 7.4, 0.05% Tween20) before application to the Fusion 5 membrane strip. The beads prevent migration of the anti-FITC antibodies through the membrane. The strip was dried at 37° C. for 30 minutes prior to use.

### Assaying on the Lateral Flow Device

The PCR product sample was prepared for assaying by taking a 10 μl aliquot and mixing it with 160 μl buffer (PBS-0.05% Tween20) and 100 of gold microparticles (OD530=10.1), with goat anti-biotin antibodies pre-adsorbed to the microparticle surface (Alchemy Laboratories). The sample was immediately loaded directly onto the Fusion 5 membrane strip of the lateral flow strip. The mixture wicked laterally through the membrane and reached the test line after approximately 10 minutes.

### Results and Discussion

The sample produced a pinkish-red line at the test line indicating a positive Listeria result. The result indicated that the use of a single layer porous matrix material such as Fusion 5 in the lateral flow device is a viable alternative to other membrane types such as nitrocellulose. The use of a single layer porous matrix material may offer advantages in terms of manufacturing.

### Example 4

### Detection of Listeria monocytogenes Using Multiplex PCR Amplification to Provide a PCR Control

### Materials and Methods

### Sample

A sample can be obtained from the third tube of a TECRA® UNIQUE PLUS™ Listeria test module.

### Control Template

A control template (and complementary strand) of a non-related nucleotide sequence (i.e. a nucleotide sequence not found in L. monocytogenes) can be synthesised according to methods well known to persons skilled in the art. A suitable control template prepared from a control nucleic acid of the platypus mannose 6-phosphate/insulin-like growth factor 2 receptor (M6P/IGF-2R) gene, has the sequence:

### Coding Strand:

(SEQ ID NO: 3)

5′-TTGAAAGACGATGAAGAAAGTAAGCCAGATTTCTGCAATGGCCATAA
TCCAGCAGTGACCATTACATTTATATGCCCGTCAGGGAGAATAGAAAGCA
CAGCTCCCAAGCTCACGGCTAAATCCAACTGCCGGTATGAGGTGGAGTGG
ATCACTGAGTACACCTGCCATAGAGATTATTTGGAAAGTAATTCCTGCTA
TCTAAATAG-3′

### Non-Coding Complementary Strand:

(SEQ ID NO: 4)

5′-CTATTTAGATAGCAGGAATTACTTTCCAAATAATCTCTATGGCAGGG
TACTCAGTGATCCACTCCACCTCATACCGGCAGTTGGATTTAGCCGTGAG
CTTGGGAGCTGTGCTTTCTATTCTCCCTGACGGGCATATAAATGTAATGG
TCACTGCTGGATTATGGCCATTGCAGAAATCTGGCTTACTTTCTTCATCG
TCTTTCAA-3′

### PCR Primers

Primers are selected to enable multiplex PCR amplification of a region of L. monocytogenes, for example, a 130 by region of the invasion associated protein (IAP) gene, and a 206 base pair region of a control nucleic acid (i.e. the 938-1143 by region of the platypus M6P/IGF-2R gene; Genbank Accession No AF151172). The nucleotide sequences of suitable primers are:

1st Primer Pair (IAP Gene, L. monocytogenes)

(SEQ ID NO: 5)

| | |
|---|---|
| Forward primer: | 5′-ACAAGCTGCACCTGCTGCAG-3′ |

(SEQ ID NO: 6)

| | |
|---|---|
| Reverse primer: | 5′-TAACAGCGTGTGTAGTAGCA-3′ |

The primers of the first primer pair are labelled at the 5′ end; with biotin for the forward primer, and with fluorescein for the reverse primer.

### 2nd Primer Pair (M6P/IGF-2R Gene)

(SEQ ID NO: 7)

| | |
|---|---|
| Forward primer: | 5′-TTGAAAGACGATGAAGAAAGTAAG-3 |

(SEQ ID NO: 8)

| | |
|---|---|
| Reverse primer: | 5′-CTATTTAGATAGCAGGAATTACTTTC-3′ |

The primers of the second primer pair are labelled at the 5′ end; with biotin for the forward primer, and with dinitrophenol (DNP) for the reverse primer.

### Lateral Flow Device

A lateral flow device as described in Example 1 is prepared. The test line is prepared by adsorbing anti-FITC antibodies to the membrane in a thin line across the width of the membrane in the test zone. A PCR control line is preferably placed between the test line and the distal end of the device. The PCR control line is prepared by adsorbing anti-DNP antibodies in a thin line across the width of the membrane.

### Amplification

PCR amplification can be conducted in accordance with standard methods, however all four primers and a small amount (e.g. 10-100 copies) of control template are added to the mixture. With the primers described above (i.e. SEQ ID NO: 5, 6, 7 and 8), the PCR amplification can be conducted as follows:
(i) Rehydrate dried PCR mix (Accupower, Bioneer, Korea) using 20 μmolecular quality H₂O comprising a final concentration of 250 nM of each primer (SEQ ID NO: 5, 6, 7 and 8) and 10-100 copies of double stranded positive control template DNA (SEQ ID NO: 3 and 4);
(ii) Inoculate 1 μl of sample using a sterile inoculation loop into the rehydrated dried PCR mix;
(iii) Using a standard thermal cycler PCR machine, subject the inoculated PCR mix to an initial heating step of 94° C. for 5 minutes; followed by
(iv) 40 cycles of
   a. melting step, 94° C. for 15 seconds,
   b. annealing step, approximately 58° C. for 20 seconds, and
   c. elongation step, 72° C. for 30 seconds.

### Preparation of PCR Product

A 10 μl aliquot of the PCR product is mixed with 120 μl running buffer comprising phosphate buffered saline (PBS, pH 7.5) and Tween-20 (0.05%), and 10 μl of gold microparticles (OD530=10.2) onto which goat anti-biotin antibodies have been pre-adsorbed (Alchemy Laboratories).

### Assaying on the Lateral Flow Device

A 140 μl aliquot of the buffered PCR product mixture is loaded onto the lateral flow device. The constituents of the mixture are allowed to flow across the membrane for 1 to 10 minutes. The test line comprising anti-biotin antibodies traps any L. monocytogenes amplicons present in the mixture that were doubly labelled with biotin and FITC. The PCR control line of anti-DNP traps amplicons generated from the control template that were doubly labelled with biotin and DNP.

### Example 5

### Detection of Listeria monocytogenes Using Two Tube Control PCR Amplification

### Materials and Methods

### Sample

A sample can be obtained from the third tube of a TECRA® UNIQUE PLUS™ Listeria test module.

### PCR Primers

Primers are selected to enable multiplex PCR amplification of a region of L. monocytogenes, for example, a 130 base pair region of the invasion associated protein (IAP) gene, and a 206 base pair region of a control nucleic acid (e.g. the 938-1143 by region of the platypus M6P/IGF-2R gene; Genbank Accession No AF151172). Suitable labelled primers are as described in Example 4.

### Lateral Flow Device

A lateral flow device as described in Example 1 is prepared. Test and PCR control lines are prepared as described in Example 4.

### Amplification

PCR amplification is conducted in two separate reaction tubes. The test reaction (i.e. the reaction using the sample) is carried out as described in Example 4 with the exception that the reaction is run in two separate tubes such that:
(i) Two tubes of dried PCR mix (Accupower, Bioneer, Korea) are rehydrated. One contains 20 μl molecular quality H₂O comprising a final concentration of 250 nM of the first primer pair (SEQ ID NO: 5 and 6) and the second tube contains 20 μl molecular quality H₂O comprising 250 nM of the second primer pair (SEQ ID NO: 7 and 8) and 10-100 copies of double stranded positive control template DNA (SEQ ID NO: 3 and 4);
(ii) Inoculate 1 μl of sample using a sterile inoculation loop into each of the tubes of rehydrated dried PCR mix;
(iii) Using a standard thermal cycler PCR machine, subject the inoculated PCR mix to an initial heating step of 94° C. for 5 minutes; followed by
(iv) 40 cycles of
   a. melting step, 94° C. for 15 seconds,
   b. annealing step, approximately 58° C. for 20 seconds, and
   c. elongation step, 72° C. for 30 seconds.

### Preparation of PCR Product

A 5 μl aliquot of each PCR product is mixed together with 120 μl running buffer (phosphate buffered saline, pH 7.5 and 0.05% Tween-20), and 10 μl of gold microparticles (OD530=10.2) to which goat anti-biotin antibodies have been pre-adsorbed (Alchemy Laboratories).

### Assaying on the Lateral Flow Device

A 140 μl aliquot of the buffered PCR product mixture is loaded onto a lateral flow device. The constituents of the mixture are allowed to flow across the membrane for 1 to 10 minutes. The test line comprising anti-biotin antibodies traps any test Listeria monocytogenes amplicons present in the mixture that were doubly labelled with biotin and FITC. The PCR control line, on the other hand, captures the control amplicon.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product or step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region provided with a second agent which specifically binds to said second label and said control region being provided with a control agent; and
(v) detecting any binding of constituents of the buffered product at said test region and at said control region.

2. A method for the detection of a micro-organism present in a sample, said method comprising the steps of:
(i) treating said sample so as to cause release of nucleic acid from any of said micro-organism present in the sample;
(ii) amplifying a target nucleotide sequence present on said nucleic acid, said target sequence being unique or otherwise characteristic of said micro-organism, comprising the use of a pair of first and second primer sequences defining the 5′ and 3′ ends of said target sequence, wherein the amplification of the target sequence utilises deoxyribonucleotide triphosphates (dNTPs) labelled with a first label and said second primer sequence is labelled with a second label, such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying at least a portion of the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to the other of said first and second labels which is not bound by said first agent and said control region being provided with a control agent; and
(v) detecting any binding of constituents of the buffered product at said test region and at said control region.

3. The method according to claim 1 or 2, wherein the sample is a food sample, a sample prepared from a swab of a food preparation surface, a waste or process water sample, or a micro-organism culture or enrichment sample.

4. The method of claim 1 or 2, wherein the treating step (i) comprises heating the sample at a temperature in the range of 85 to 100° C.

5. (canceled)

6. The method of claim 1 or 2, wherein the amplification step (ii) comprises nested polymerase chain reaction (PCR) amplification.

7. (canceled)

8. The method of claim 1 or 2, wherein the first label is biotin and the second label is dinitrophenol (DNP).

9. The method claim 1 or 2, wherein at least one of said first and second primer sequences is a degenerate primer sequence.

10-14. (canceled)

15. The method of claim 1 or 2, wherein the sequences of the first and second primer sequences are family-specific.

16. The method of claim 15, wherein the sequences of the first and second primer sequences are specific to a family selected from Listeriaceae, Enterobacteriaceae, Staphylococcaceae, Bacillaceae, Legionellaceae, Pseudomonadaceae, Campylobacteraceae and Helicobacteraceae.

17. The method of claim 1 or 2, wherein the sequences of the first and second primer sequences are genus-specific.

18. The method of claim 17, wherein the sequences of the first and second primer sequences are specific to a genus selected from Listeria, Salmonella, Enterobacter, Escherichia, Legionella, Bacillus, Pseudomonas, Staphylococcus, Campylobacter and Helicobacter.

19. (canceled)

20. The method of claim 1 or 2, wherein the sequences of the first and second primer sequences are specific to Listeria monocytogenes.

21. The method of claim 1 or 2, wherein the sequences of the first and second primer sequences are specific to Enterobacter sakazakii.

22. The method of claim 1 or 2, wherein the sequences of the first and second primer sequences are specific to a species, and wherein the amplifying step (ii) further comprises amplification of a further target nucleotide sequence through the use of a pair of third and fourth primer sequences defining the 5′ and 3′ ends of said further target sequence, said third and fourth primer sequences being specific for the genus to which the said species belongs and labelled with, respectively, third and fourth labels, such that any amplification of the target sequence and further target sequence generates a species-specific amplicon labelled with both first and second labels and/or a genus-specific amplicon labelled with both the third and fourth labels, wherein said third and fourth labels either both differ from the first and second labels or, alternatively said third label is the same or functionally equivalent to the first label and said fourth label differs from the first and second labels.

23. The method of claim 22, wherein the sequences of the first and second primer sequences are specific to Listeria monocytogenes.

24. The method of claim 23, wherein the sequences of the third and fourth primer sequences are specific to the genus Listeria.

25. The method of claim 1 or 2, wherein the sequences of the first and second primer sequences are specific to a first genus, and wherein the amplifying step (ii) further comprises amplification of a further target nucleotide sequence through the use of a pair of third and fourth primer sequences defining the 5′ and 3′ ends of said further target sequence, said third and fourth primer sequences being specific for a second genus and labelled with, respectively, third and fourth labels, such that any amplification of the target sequence and further target sequence generates an amplicon labelled with both first and second labels and/or an amplicon labelled with both the third and fourth labels, wherein said third and fourth labels either both differ from the first and second labels or, alternatively, said third label is the same or functionally equivalent to the first label and said fourth label differs from the first and second labels.

26. The method of claim 25, wherein the sequences of the first and second primer sequences are specific to the genus Listeria.

27. The method of claim 26, wherein the sequences of the third and fourth primer sequences are specific to the genus Salmonella.

28. The method of claim 22, wherein at least one of said third and fourth primer sequences is a degenerate primer sequence.

29-30. (canceled)

31. The method of claim 1 or 2, wherein the amplifying step (ii) further comprises amplification of a control nucleotide sequence comprising the nucleotide sequence of SEQ ID NO: 3 and/or SEQ ID NO: 4 through the use of a pair of third and fourth primer sequences defining the 5′ and 3′ ends of said control sequence, said third and fourth primer sequences being labelled with, respectively, third and fourth labels, such that any amplification of the target sequence and control sequence generates an amplicon labelled with both first and second labels and/or an amplicon labelled with both the third and fourth labels, wherein said third and fourth labels either both differ from the first and second labels or, alternatively, said third label is the same or functionally equivalent to the first label and said fourth label differs from the first and second labels, and wherein the control region on said lateral flow device is provided with a control agent which specifically binds to the fourth label.

32-35. (canceled)

36. A kit for the detection of a micro-organism present in a sample, said kit comprising:
a pair of first and second primer sequences defining 5′ and 3′ ends of a target nucleotide sequence that is unique or otherwise characteristic of said micro-organism, said first primer sequence being labelled with a first label and said second primer sequence being labelled with a second label;
a control nucleic acid and a pair of primer sequences defining the 5′ and 3′ ends of a control nucleotide sequence, wherein the control nucleic acid comprises the nucleotide sequence of SEQ ID NO: 3 and/or SEQ ID NO: 4;
a buffer solution comprising microparticles labelled with a first agent which specifically binds to said first label; and
a lateral flow device comprising a substrate with a test region and a control region, said test region being provided with a second agent which specifically binds to said second label and said control region being provided with a control agent.

37. A kit for the detection of a micro-organism present in a sample, said kit comprising:
deoxyribonucleotide triphosphates (dNTPs) labelled with a first label;
a pair of first and second primer sequences defining 5′ and 3′ ends of a target nucleotide sequence that is unique or otherwise characteristic of said micro-organism, said second primer sequence being labelled with a second label;
a control nucleic acid and a pair of primer sequences defining the 5′ and 3′ ends of a control nucleotide sequence, wherein the control nucleic acid comprises the nucleotide sequence of SEQ ID NO: 3 and/or SEQ ID NO: 4;
a buffer solution comprising microparticles labelled with a first agent which specifically binds to said first label; and
a lateral flow device comprising a substrate with a test region and a control region, said test region being provided with a second agent which specifically binds to said second label and said control region being provided with a control agent.

38-39. (canceled)

40. A method for the detection of a nucleic acid in a sample, said method comprising the steps of:
(i) heating said sample at a temperature in the range of 85 to 100° C. so as to cause release of nucleic acid from any cell or other nucleic acid-containing structure present in the sample;
(ii) amplifying a target nucleotide sequence present on said nucleic acid, comprising the use of a pair of first and second primer sequences defining the 5′ and 3′ ends of said target sequence, said first primer sequence being labelled with a first label and said second primer sequence being labelled with a second label such that any amplification of the target sequence generates an amplicon labelled with both first and second labels;
(iii) diluting an amount of the amplification product of step (ii) in a suitable buffer solution comprising microparticles labelled with a first agent which specifically binds to said first label and allowing said first agent to bind to said first label present;
(iv) applying at least a portion of the buffered product of step (iii) at or adjacent to a proximal end of a lateral flow device comprising a substrate which—allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to said second label and said control region being provided with a control agent; and
(v) detecting any binding of constituents of the buffered product at said test region and at said control region.

41. A method for the detection of a nucleic acid in a sample, said method comprising the steps of:
(i) heating said sample at a temperature in the range of 85 to 100° C. so as to cause release of nucleic acid from any cell or other nucleic acid-containing structure present in the sample;
(ii) amplifying a target nucleotide sequence present on said nucleic acid, comprising the use of a pair of first and second primer sequences defining the 5′ and 3′ ends of said target sequence, wherein the amplification of the target sequence utilises deoxyribonucleotide triphosphates (dNTPs) labelled with a first label and said second primer is labelled with a second label, such that any amplification of the target sequence generates an amplicon labelled with both first and second labels;
(iii) diluting an amount of the amplification product of step (ii) in a suitable buffer solution comprising microparticles labelled with a first agent which specifically binds to one of said first and second labels and allowing said first agent to bind to said one of said first and second labels present;
(iv) applying the buffered product of step (iii) at or adjacent to a proximal end of a lateral flow device comprising a substrate which allows constituents of said buffered product to wick or flow laterally towards a distal end of the lateral flow device, wherein at a location at or adjacent to said distal end, the lateral flow device is provided with a test region and a control region, said test region being provided with a second agent which specifically binds to the other of said first and second labels which is not bound by said first agent and said control region being provided with a control agent; and
(v) detecting any binding of constituents of the buffered product at said test region and at said control region.

42-44. (canceled)

45. The method of claim 25, wherein the amplification step (ii) comprises nested polymerase chain reaction (PCR) amplification.

46-53. (canceled)

54. The method of claim 25, wherein the sequences of the first and second primer sequences are family-specific.

55-57. (canceled)

58. The method of claim 25, wherein the amplifying step (ii) further comprises amplification of a control nucleotide sequence comprising the nucleotide sequence of SEQ ID NO: 3 and/or SEQ ID NO: 4 through the use of a pair of third and fourth primer sequences defining the 5′ and 3′ ends of said control sequence, said third and fourth primer sequences being labelled with, respectively, third and fourth labels, such that any amplification of the target sequence and control sequence generates an amplicon. labelled with both first and second labels and/or an amplicon labelled with both the third and fourth labels, wherein said third and fourth labels either both differ from the first and second labels or, alternatively, said third label is the same or functionally equivalent to the first label and said fourth label differs from the first and second labels, and wherein the control region on said lateral flow device is provided with a control agent which specifically binds to the fourth label.

59-62. (canceled)
